(19) United States
(12) Patent Application Publication
(10) Pub. No.: US 2008/0262047 A1
(43) Pub. Date: Oct. 23, 2008

(54) Ppar-Gamma Agonists for Improvement of Cognitive Function in Apoe4 Negative Patients

(76) Inventors: Allen D Roses, Durham, NC (US); Ann M. Saunders, Durham, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/067,374

(22) PCT Filed: Sep. 20, 2006
(86) PCT No.: PCT/US06/36603

§ 371 Date: Mar. 19, 2008

Related U.S. Application Data
(60) Provisional application No. 60/719,353, filed on Sep. 22, 2005.
(60) Provisional application No. 60/727,377, filed on Oct. 17, 2005.

(51) Int. Cl. A61K 31/425; A61K 31/44; A61P 25/00

(52) U.S. Cl. 514/342; 514/369

(57) ABSTRACT

Disclosed is a method for improving cognitive function in a subject suffering from or susceptible to MCI, Alzheimer's disease or other dementias, which subject is not homozygous for the APOE4 allele, comprising the steps of:
(i) screening the subject to determine that the subject is not homozygous for the APOE4 allele; and then
(ii) administering a safe and effective amount of a PPAR-gamma agonist to said subject.

The present invention relates to the treatment or prevention of mild cognitive impairment and Alzheimer's disease as well as other dementias and in particular to the improvement of cognitive function therein.

Alzheimer's disease (AD) was first described in 1907 by the Bavarian psychiatrist Alois Alzheimer. It is a progressive, debilitating disease and is the most common cause of dementia. Typical symptoms include memory impairment, disordered cognitive function, behavioural changes (including paranoia, delusions, loss of inhibitions) and decline in language function. Pathologically, AD has been traditionally characterised by the presence of two distinct types of brain lesion—neuritic plaques (sometimes referred to as senile plaques) and neurofibrillary tangles.

Neuritic plaques are extracellular amyloid β-protein (Aβ) deposits, typically in a filamentous form, which are around 10 to 150 μm in cross-section and are associated with axonal and dendritic injury. Aβ is formed by the cleavage of amyloid precursor protein (APP) by a series of secretases. Aβ₄₀, a forty residue peptide, is the form of Aβ normally produced in greatest abundance by cells, however, much of the Aβ found within neuritic plaques contains 42 amino acids (Aβ₄₂). Aβ₄₂ is significantly more hydrophobic than Aβ₄₀, and is therefore more prone to aggregation, although Aβ₄₀ is also localised with the plaques. Neuritic plaques are believed to develop over a substantial period of time (months to years). Amyloid depositions in the form of plaques are known to occur prior to the appearance of clinical symptoms, though the correlation between the extent of amyloid deposition and cognitive impairment remains a point of contention.

Neurofibrillary tangles are usually found within the perinuclear cytoplasm of neurons from AD sufferers. The tangles are formed from pairs of filaments which are wound into helices. These highly insoluble filaments have been shown to be composed of the microtubule-associated protein tau in an abnormally hyperphosphorylated state. There is some evidence that the formation of tangles is a response by neurons to the gradual accumulation of Aβ.

Clinically typical AD can be inherited in an autosomal dominant manner however, most cases of the disease (approximately 90%) are considered to be sporadic. These two forms of the disease are phenotypically highly similar save that the rarer familial AD generally presents much earlier than sporadic AD (as such, often known as late-onset AD or LOAD). This general phenotypic similarity suggests that information characterising the mechanism underlying autosomal dominant forms (such as mutations in APP and the presenilin 1 and 2 genes) has relevance to the late-onset sporadic form of AD. Generally, familial AD is associated with increased production of Aβ, whereas sporadic AD may be the result of defective clearance of regular Aβ production.

A large number of contributory factors have been identified for sporadic AD, including: age, low cholesterol concentration, high systolic blood pressure, high glucose concentrations, high insulin concentrations, abnormal glucose tolerance and the presence of an e4 allele of Apolipoprotein E (Kuusisto J et al. BMJ 1997 315:1045-1049).

For further information on AD in general see: Selkoe D Physiol. Rev. 2001 81 (2):741-766; Watson G et al. CNS Drugs 2003 17(1):27-45.

Mild cognitive impairment is a condition in which subjects have a slight impairment in cognitive function that is detectable from their pre-morbid baseline, but which also is not sufficiently severe to fulfil diagnostic criteria for AD. As such, MCI may be considered as a transition state between normal cognitive function in a normal aging subject, and the abnormal cognitive function in dementia. MCI can be subdivided into categories based upon the types of cognitive deficits that are detected. A deficit of memory alone typifies amnestic MCI; whereas other types of MCI involve deficits in multiple cognitive domains including memory, or deficits in a single, non-memory domain. The rate of progression from amnestic MCI to AD has been measured in cohort studies to range from 10-20% per year (for more information see Petersen et al. Arch Neurol 2001 58: 1985-1992).

Other dementias which similarly give rise to cognitive deficits include vascular dementia, Lewy body dementia, frontotemporal dementia and dementia associated with Parkinson's disease.

Apolipoproteins are glycoproteins which have been associated with brain development, synaptogenesis and response to neuronal injury. Apolipoprotein E (ApoE) is one protein component of plasma lipoproteins. There are three major isoforms of ApoE (i.e. ApoE2, ApoE3 and ApoE4), which are products of three alleles at a single gene locus. Individuals may therefore be homozygous (APOE2/2, APOE3/3 or APOE4/4) or heterozygous (APOE2/3, APOE2/4 or APOE3/4). The most common allele is APOE3, having an allele frequency in the Caucasian population of approximately 0.78 (Bales K R et al. Mol. Interventions. 2002 2: 363-375), and the most common genotype is APOE3/3.

The amino acid sequence of the three isoforms show only slight variation, which is summarised in the Table 1 below.

TABLE 1
Amino-acid sequence variation in apolipoprotein isoforms.

| | ApoE2 | ApoE3 | ApoE4 |
|---|---|---|---|
| Residue 112 | Cysteine | Cysteine | Arginine |
| Residue 158 | Cysteine | Arginine | Arginine |

An association between carriage of an APOE4 allele and the risk of developing AD has been known for some time and is well documented in the literature (Strittmafter W J et al. PNAS 1993 90:1977-1981; Roses A D Ann Rev Med 1996 47: 387-400). However, APOE genotyping alone is not a sufficient diagnostic test for AD since the presence of the e4 allele is a susceptibility factor and does not cause the disease (Mayeux R et al. New Engl. J. Med. 1998 338:506-511).

The age-adjusted risk of AD in individuals having two APOE4 alleles has been shown to be over three times that of individuals having only one APOE4 allele, which is in turn almost three times that of individuals who do not have an APOE4 allele (Corder et al. Science 1993 261(5123):921-3; Kuusisto J et al. BMJ 1994 309:636-638). Relative to other AD patients, those which are homozygous for APOE4 show an earlier age of onset, increased amyloid burden and decreased acetylcholine levels. The APOE4 allele frequency varies across ethnic populations and has been found to be approximately 0.15 in the Caucasian population but up to 0.4 in patients with AD (Saunders et al. Neurology 1993 43(8): 1467-72).

APOE2, the rarest of the three common alleles, has been suggested to have a protective effect relative to the most common APOE3 allele, individuals having an APOE2 allele generally showing a later onset of disease than those without (Corder et al. Nature Genetics 1994 7(2):180-4; Bales K R et al. Mol Interventions 2002 2: 363-375). The APOE2 allele frequency has been found to be approximately 0.07 in the Caucasian population. There are more recent data that APOE4 status has no bearing on rate of progression once symptoms of possible AD are present.

Glucose metabolism is of critical importance in the function of cells within the central nervous system. Decreases in cerebral glucose metabolism that are regionally specific have been demonstrated in patients with AD (Reiman E M et al. New Eng J Med 1996 334: 752-758; Alexander, G E et al. Am J Psychiatry 2002 159:738-745), both in LOAD and in familial AD (Small G W et al., PNAS 2000 97: 6037-6042).

The decrease in cerebral glucose metabolism in patients at risk for AD has been linked to APOE status, because the regionally specific pattern of decreased cerebral glucose metabolism can be detected many years before the predicted age of onset of clinical symptoms, in individuals who carry one or two APOE4 alleles (Reiman E M et al. New Eng J Med 1996 334: 752-758; Rossor M et al., Annals NY Acad Sci 1996 772:49-56; Small G W et al., PNAS 2000 97: 6037-6042).

Insulin is also of critical importance in peripheral and central energy metabolism. Secreted by pancreatic α-cells, plasma insulin serves to regulate glucose levels in the blood through periods of feeding and fasting, the rate of glucose uptake in insulin sensitive tissues being controlled by insulin-sensitive glucose transporters.

Increases in blood glucose result in the release of insulin, while decreases in blood glucose results in the release of counter-regulatory hormones which increase glucose output by the liver. Type II diabetes results from a reduced ability of insulin to stimulate glucose uptake and to inhibit hepatic glucose output (known as insulin resistance) and an insufficient insulin secretory response to compensate for the insulin resistance.

Insulin is transported across the blood/brain barrier by an insulin receptor-mediated transport process. Peripheral levels of insulin tend to correlate with levels in the central nervous system (CNS), i.e. increased peripheral insulin results in increased CSF insulin. Evidence suggests that insulin has some involvement in normal memory function, and that disorders in peripheral insulin metabolism, such as insulin resistance and hyperinsulinaemia, may have a negative influence on memory. Insulin-promoted increases in glucose utilisation may lead to glycolytic production of acetyl-CoA, the key substrate in the synthesis of the neurotransmitter acetylcholine. Reduction in acetylcholine levels is a key feature of AD.

Peroxisome Proliferator-Activated Receptor gamma (PPAR-gamma) is an orphan member of the steroid/thyroid/retinoid receptor superfamily of ligand-activated transcription factors. PPAR-gamma is one of a subfamily of closely related PPARs encoded by independent genes (Dreyer C et. al. Cell 1992 68:879-887; Schmidt A et al. Mol. Endocrinol. 1992 6:1634-1641; Zhu et al. J. Biol. Chem. 1993 268:26817-26820; Kliewer S A et al. Proc. Nat. Acad. Sci. USA 1994 91:7355-7359). Three mammalian PPARs have been isolated and termed PPAR-alpha, PPAR-gamma, and PPAR-delta (also known as NUC-1). These PPARs regulate expression of target genes by binding to DNA sequence elements, termed PPAR response elements (PPRE). To date, PPREs have been identified as the enhancers of a number of genes encoding proteins that regulate lipid metabolism, suggesting that PPARs play a pivotal role in the adipogenic signalling cascade and lipid homeostasis (Keller H et al. Trends Endocrin. Met. 1993 4:291-296).

European Patent 306228 describes a class of PPAR-gamma agonists which are thiazolidinedione derivatives for use as insulin sensitisers in the treatment of Type II diabetes mellitus. These compounds have anti-hyperglycaemic activity. One preferred compound described therein is known by the chemical name 5-[4-[2-(N-methyl-N-(2-pyridyl)amino)ethoxy]benzyl]thiazolidine-2,4-dione and has been given the generic name rosiglitazone. Salts of this compound, including the maleate salt, are described in WO94/05659. European Patent Applications, Publication Numbers: 0008203, 0139421, 0032128, 0428312, 0489663, 0155845, 0257781, 0208420, 0177353, 0319189, 0332331, 0332332, 0528734, 0508740; International Patent Applications, Publication Numbers 92/18501, 93/02079, 93/22445 and U.S. Pat. Nos. 5,104,888 and 5478852, also disclose certain thiazolidinedione PPAR-gamma agonists. Specific compounds that may be mentioned include 5-[4-[2-(5-ethyl-2-pyridyl)ethoxy]benzyl]thiazolidine-2,4-dione (also known as pioglitazone), 5-[4-[(1-methylcyclohexyl)methoxy]benzyl]thiazolidine-2,4-dione (also known as ciglitazone), 5-[[4-[(3,4-dihydro-6-hydroxy-2,5,7,8-tetramethyl-2H-1-benzopyran-2-yl)methoxy]phenyl]methyl]-2,4-thiazolidinedione (also known as troglitazone) and 5-[(2-benzyl-2,3-dihydrobenzopyran)-5-ylmethyl)thiazolidine-2,4-dione (also known as englitazone).

U.S. Pat. No. 6,294,580 (the disclosure of which is herein incorporated by reference) describes a series of PPAR gamma agonist compounds not of the thiazolidinedione class but which are instead O- and N-substituted derivatives of tyrosine which nevertheless are effective as insulin sensitisers in the treatment of Type II diabetes mellitus. One such compound has chemical name N-(2-benzoylphenyl)-O-[2-(5-methyl-2-phenyl-4-oxazolyl)ethyl]-L-tyrosine (also known as 2(S)-(2-Benzoyl-phenylamino)-3-{4-[2-5-methyl-2-phenyl-oxazol-4-yl)-ethoxy]-phenyl}-propionic acid, or by the generic name farglitazar).

A body of clinical evidence suggests that impairment of cerebral glucose metabolism is present during AD, and in APOE4-carriers before the clinical onset of symptoms of AD (Reiman E M et al. New Eng J Med 1996 334: 752-758; Rossor M et al., Annals NY Acad Sci 1996 772:49-56; Small G W et al., PNAS 2000 97: 6037-6042).

Converging clinical and epidemiological evidence also suggests that the risk of developing AD may be influenced by insulin resistance. However, the exact nature of the relationship between insulin resistance and AD is complex and not presently fully understood.

Hyperinsulinaemia has been shown to be a risk factor for AD. In one study it was concluded by the authors to be independent of APOE genotype (Kuusisto J et al. BMJ 1997 315:1045-1049), where hyperinsulinaemic elderly subjects without an APOE4 allele (APOE4−) had an AD prevalence of 7.5% in hyperinsulinaemic subjects, compared with 1.4% in normoinsulinaemic subjects; while hyperinsulinaemic elderly subjects with an APOE4 allele (APOE4+) had an AD prevalence of 7.0% hyperinsulinaemic subjects, compared with 7.1% in normoinsulinaemic subjects. Other studies have indicated a link between APOE genotype and insulin resistance (Watson G et al. CNS Drugs 2003 17(1):27-45).

For example, patients who were not homozygous for the APOE4 allele have abnormalities of insulin metabolism (specifically increased plasma insulin levels), suggesting a possible factor in the development of AD in these patients, while those who were homozygous for the APOE4 allele demonstrated normal peripheral levels of insulin. Both groups demonstrated reduced cerebrospinal fluid insulin levels compared to non-AD subjects (Craft S et al. Neurology 1998 50:164-168). Furthermore, patients without an APOE4 allele have reduced rates of insulin-mediated glucose disposal relative to those who are APOE4+ (Craft S et al. Neuroendocrinology 1999 70:146-152).

It is well accepted that normal cholinergic signalling is a necessity for the proper function of mental processes such as memory. A large body of evidence indicates that AD patients have abnormalities in cholinergic signalling, the extent of which correlates with the level of cognitive impairment. As with many aspects of AD research the link between the progression of the disease and the observed cholinergic dysfunction is not fully understood. To date the use of agonists for muscarinic or nicotinic acetylcholine receptors has not proved to be of clinical value, though a number of cholinesterase inhibitors have demonstrated sufficient efficacy with an acceptable degree of adverse effects to be approved for use in the treatment of AD, these include tacrine (Cognex™), galantamine (Reminyl/Radazyne™), rivastigamine (Exelon™) and donepezil (Aricept™). For further information see, for example, Terry A V et al. J. Pharmacol. Exp. Ther. 2003 306(3):821-827.

In a recently published study investigating the use of donepezil in individuals with mild cognitive impairment (a transitional state between normal aging and early AD), donepezil was shown to reduce the rate at which patients developed AD during the first twelve months of administration, although at three years there was no separation between groups. Additionally, the beneficial effect seen in the first 12 months was then followed by a more acute deterioration in the following 24 months. Although no significant difference in the general intent to treat population was observed after three years compared to placebo, patients who were carriers of one or two copies of an APOE4 allele had a reduced risk of progressing to AD compared to placebo (Petersen R et al. New Engl. J. Med. 2005 352:2379-2387).

Over-stimulation of the N-methyl-D-aspartate (NMDA) receptor by glutamate is thought to contribute to the pathogenesis of AD. NMDA receptor antagonists are therefore a further class of compounds which are of use in the clinical treatment of AD: memantine (Axura™, Namenda™) is the first NMDA receptor antagonist to be approved by the FDA. Based around an adamantane core, memantine has been shown to significantly retard the rate of deterioration in patients with moderate to severe AD while having a low incidence of adverse effects (Resiberg B et al. New Engl. J. Med. 2003 348:1333-1341). There are more recent data that the mechanism of action of memantine is not NMDA-blockage alone but may also involve effects on the a7 nicotinic acetylcholine receptor (Aracava et al J Pharmacol Exper Therapeutics, 2005 312(3): 1195-1205).

At a cellular and molecular level a number of inflammatory processes may be observed in the brains of AD sufferers, and these inflammatory processes are considered to be of importance in the development and progression of the disorder. There is some evidence that non-steroidal anti-inflammatory drugs (NSAIDs) may lower the risk of AD, slow the progression of the disease and reduce the severity of cognitive symptoms (in t′ Veld B A et al. Epidemol. Rev. 2002 24(2):248-268; Etminan M et al. BMJ2003 327:128-132). However, clinical trials have yet to be successfully completed due to an unexpected occurrence of cardiovascular effects in trial subjects. One clinical trial using rofecoxib was completed for AD and MCI (Reines et. al. Neurology 2004 62: 66-71) but failed to show any efficacy.

The use of insulin sensitizers in the treatment of AD has been proposed previously. International patent application WO98/39967 discloses a method for the treatment or prevention of AD by administering an agent which reduces serum insulin levels, such as a thiazolidinedione. International patent application WO99/25346 discloses a method for the treatment or prevention of a disease mediated by apoptosis, such as neurodegenerative disorders including AD and Parkinson's disease by administering an apoptosis inhibitor, for example an insulin sensitising agent such as rosiglitazone. International patent application WO00/32190 discloses a method for the treatment or prevention of AD by administering a PPAR-gamma agonist, such as the thiazolidinediones pioglitazone and rosiglitazone. International patent application WO00/35437 discloses methods of improving mental performance in subjects suffering from reduced mental performance by the administration of insulin sensitising agents, such as the thiazolidinediones pioglitazone and rosiglitazone.

In Parkinson's disease models, there is evidence that thiazolidinediones (including rosiglitazone and pioglitazone) can protect dopaminergic cells from various toxic insults including acetaldehyde (Jun et al (2006) Biochem Biophys Res Comm 340, 221-227), MPTP (Dehmer et al (2004) J Neurochem 88, 494-501) and 8-OHDA (Chen et al (2004) FASEB 18, 1162-1164).

Prior to the earliest priority date of this patent application, there has been no definitive evidence which shows that the use of PPAR-gamma agonists to improve cognitive function in subjects suffering from or susceptible to MCI, AD or other dementias provides benefit only to those subjects who are not homozygous for the APOE4 allele, and provides most benefit to those who are non-carriers of the APOE4 allele.

### BRIEF DESCRIPTION OF THE FIGURES

FIG. 1 shows the model adjusted ADAS-cog change from baseline in the intent to treat population of Example 2.

FIG. 2 shows the model adjusted ADAS-cog change from baseline in the genotyped population of Example 2 by treatment regime and APOE allele status.

FIG. 3 shows a plot of model adjusted ADAS-cog change from baseline in the APOE4 heterozygote (“Het”) and APOE4 homozygote (“Homo”) populations of Example 2.

According to the present invention there is provided a method for improving cognitive function in a subject suffering from or susceptible to MCI, Alzheimer's disease or other dementias, which subject is not homozygous for the APOE4 allele, comprising the steps of:
(i) screening the subject to determine that the subject is not homozygous for the APOE4 allele; and then
(ii) administering a safe and effective amount of a PPAR-gamma agonist to said subject.

In one embodiment of the invention, screening step (i) involves determining that the subject carries a single copy of the APOE4 allele. For example the subject may be determined to be APOE3/APOE4.

In a more preferred embodiment of the invention screening step (i) involves determining that the subject is APOE4− (i.e. does not carry the APOE4 allele). Screening step (i) may, for instance, comprise determining whether the subject has an APOE2 or an APOE3 allele. For example the subject may be determined to be APOE3/APOE3 or APOE2/APOE3.

For example the subject may be suffering from or be susceptible to (for example may be suffering from) MCI or AD. In one embodiment of the invention the subject is suffering from MCI (particularly amnestic MCI). In another embodiment of the invention the subject is suffering from Alzheimer's disease. In another embodiment of the invention the subject is susceptible to MCI (particularly amnestic MCI). In another embodiment of the invention the subject is susceptible to Alzheimer's disease. In further embodiments the subject is suffering from or susceptible to other dementias such as vascular dementia, Lewy body dementia, frontotemporal dementia or dementia associated with Parkinson's disease.

Also provided according to the present invention is a method of screening a subject suffering from or susceptible to MCI, Alzheimer's disease or other dementias as an aid in predicting the subject's response to administration of a PPAR-gamma agonist, comprising screening to determine whether the subject carries zero or 1 copy of the APOE4 allele.

The method may in particular include screening to determine whether the subject is APOE4−. The screening method may, for instance, comprise determining whether the subject has an APOE2 or an APOE3 allele.

In another aspect of the present invention there is provided a PPAR-gamma agonist for use in improving cognitive function in a subject suffering from or susceptible to MCI, Alzheimer's disease or other dementias, which subject has been pre-determined not to be homozygous for the APOE4 allele. The subject may, for example, have been pre-determined to be APOE4−.

In a further aspect of the present invention there is provided the use of a PPAR-gamma agonist in improving cognitive function in a subject suffering from or susceptible to MCI, Alzheimer's disease or other dementias, which subject has been predetermined not to be homozygous for the APOE4 allele. The subject may, for example, have been pre-determined to be APOE4−.

In another aspect of the present invention there is provided the use of a PPAR-gamma agonist in the manufacture of a medicament for improving cognitive function in a subject suffering from or susceptible to MCI, Alzheimer's disease or other dementias, which subject has been pre-determined not to be homozygous for the APOE4 allele. The subject may, for example, have been pre-determined to be APOE4−.

There is also provided a method of improving cognitive function in a subject suffering from or susceptible to MCI, Alzheimer's disease or other dementias, which subject is not homozygous for the APOE4 allele, which method comprises administering a safe and effective amount of a PPAR-gamma agonist to said subject; and a PPAR-gamma agonist for use in improving cognitive function in a subject suffering from or susceptible to MCI, Alzheimer's disease or other dementias, which subject is not homozygous for the APOE4 allele; and use of a PPAR-gamma agonist in improving cognitive function in a subject suffering from or susceptible to MCI, Alzheimer's disease or other dementias, which subject is not homozygous for the APOE4 allele; and use of a PPAR-gamma agonist in the manufacture of a medicament for improving cognitive function in a subject suffering from or susceptible to MCI, Alzheimer's disease or other dementias, which subject is not homozygous for the APOE4 allele. According to a particular aspect of the invention, in said method, PPAR-gamma agonist or use, the subject is APOE4−.

There is also provided a method for improving cognitive function in a subject, comprising administering to a subject in need thereof a therapeutically effective amount of a PPAR-gamma agonist, wherein the subject is not homozygous for the APOE4 allele (eg the subject is APOE4−).

There is also provided a method for determining whether a subject having or likely to develop a disease affecting cognitive performance can be treated with a PPAR-gamma agonist, comprising determining whether a subject in need thereof has two APOE4 alleles, wherein if the subject does not have two APOE4 allele (i.e. the subject has zero or one APOE4 alleles), the subject can be treated with a PPAR-gamma agonist. A particular such method comprises determining whether a subject in need thereof has zero APOE4 alleles, wherein if the subject has zero APOE4 alleles, the subject can be treated with a PPAR-gamma agonist.

There is also provided a kit comprising (i) a PPAR-gamma agonist and (ii) instructions directing administration of the PPAR gamma agonist (typically in the form of a pharmaceutical composition) to a subject who is not homozygous for the APOE4 allele (for example a subject who has been pre-determined not to be homozygous for the APOE4 allele). For example the instructions direct administration of the PPAR gamma agonist to a subject suffering from or susceptible to MCI or Alzheimer's disease or other dementias who is not homozygous for the APOE4 allele. According to a particular aspect of the invention, the subject is APOE4− (for example the subject has been pre-determined not to have any copy of the APOE4 allele).

There is also provided a kit comprising a PPAR-gamma agonist and one or more reagents for determining whether a subject has one or two (eg two) APOE4 alleles. In such a kit the one or more reagents may be selected from the group consisting of a probe, a primer, an antibody or a combination thereof.

Alternatively in the above aspects of the invention the subject may carry, or may be determined or pre-determined to carry, a single copy of the APOE4 allele. For example the subject may be or may be determined or pre-determined to be APOE3/APOE4.

As shown in the Examples below, the inventors have unexpectedly discovered that the PPAR-gamma agonist rosiglitazone produces a clinically relevant improvement in cognitive function relative to placebo in subjects with mild to moderate AD who do not carry the APOE4 allele. The results suggest that patients who carry one copy of the APOE4 allele experience a stabilisation in cognitive function (i.e. neither significant improvement nor decline) on treatment with rosiglitazone. The results suggest that patients who are homozygous for the APOE4 allele may experience a clinical decline on treatment with rosiglitazone, although it is not clear whether or not the decline was a result of treatment or due to natural progression of the disease.

Without being limited by theory, the inventors have attempted to rationalise this invention. According to one theory, the amino acid sequence differences between the isoforms results in a difference in their protein folding. In particular ApoE2 and ApoE3 are characterised by the presence of Cys at position 112 and Arg at position 61. ApoE4 is characterised by the presence of Arg at position 112 and Arg at position 61. Residues 61 and 112 interact in the folded protein and since Arg is positively charged and Cys is negatively charged the ApoE2 and ApoE3 protein folding is tighter in this region than the ApoE4 protein folding. Although all ApoE isoforms experience intracellular degradation, it is believed that as a result of conformational differences between the isoforms ApoE4 experiences a faster rate of degradation. The fragments produced in degradation have lipid and receptor binding sites that in concert cause mitochondrial toxicity. The lipid binding site of the ApoE4 fragment appears to be a more avid binder of lipids than that of the ApoE2 or ApoE3 fragments. The ApoE4 fragment therefore binds to and disrupts mitochondria to a greater extent than the ApoE2 and ApoE3 fragments; this disruption also affects mitochondrial transport from the soma to the synapse. This disruption can also render mitochondria less responsive to increasing glucose or lactate substrate which is a consequence of treatment with PPAR-gamma agonists. The effect would be expected to be greater for subjects with 2 copies of the APOE4 allele than those with one copy and greater for subjects with one copy of the APOE4 allele than those carrying no copies.

The predetermination of whether the subject carries zero, one or two copies of the APOE4 allele may, for example, be carried out by the APOE4 screening methods described herein.

In one embodiment of the invention the subject will suffer from Type II diabetes. In another embodiment of the invention the subject will not suffer from Type II diabetes.

Procedures for the diagnostic screening of subjects to determine the presence or absence of the APOE4 allele (or the presence or absence or an APOE2 or an APOE3 allele) are well documented in the literature and are within the capabilities of one skilled in the art.

The absence of the APOE4 allele may be determined directly, by a negative result in tests which indicate the presence of the allele, or indirectly, for example by positive results in tests which indicate the presence of the APOE2 and APOE3 alleles (thereby excluding the possibility that an APOE4 allele is present).

Screening methodology may be based in a number of approaches such as isoelectric focusing methods, immunological methods, immunochemical methods or sequencing methods (either of the ApoE protein itself or of the nucleic acids encoding it). Specific methods include PCR-based methods using restriction fragment enzymes or TaqMan primers.

Immunological methods involve the detection of ApoE isoforms by the use of isoform specific antibodies. However, immunological detection methods may be hampered by problems with antibody cross-reactivity, which can impact the reliability of results.

Immunochemical methods include those described in International Patent Application WO94/09155 (related to granted patents EP0625212, JP03265577 and U.S. Pat. No. 5,508,167), which discloses methods for detecting the presence or absence of ApoE4 for the diagnosis of AD. The methods for detecting the presence or absence of ApoE4 disclosed in WO94/09155 are also of use in the practice of the present invention. Briefly, a sample from the subject (e.g. a blood sample) is contacted with a solid support designed to react specifically with sulfhydryl groups. The liquid sample is then separated from the solid support and tested for the presence of ApoE by the use of an appropriate antibody. The presence of ApoE4 in the separated sample indicates that the subject is a carrier of the APOE4 allele. Unlike ApoE2 and ApoE3, the ApoE4 protein does not contain any cysteine residues and therefore does not react with and become immobilised onto the solid support. The presence of unbound ApoE in the liquid sample after passing over the solid support indicates that the individual is ApoE4+; the absence of ApoE immunoreactivity in the liquid sample after passing over the solid support indicates that the individual is ApoE−. Issues with antibody specificity are largely negated by this approach, since it does not require the immunological differentiation of ApoE isoforms.

Sequencing approaches involve the isolation and purification of either ApoE protein or the DNA encoding ApoE from the subject, determination of the amino-acid or DNA sequence by conventional means, and comparison of the results with known amino-acid or DNA sequences for the different alleles.

The preferred method of determining APOE genotype involves using PCR-based methods—primarily PCR of a portion of the APOE gene followed by digestion with restriction enzymes that recognize the DNA substitutions that distinguish the alleles and gel electrophoresis or most currently, using TaqMan real time PCR.

Specifically, APOE genotyping may be performed using an established Taqman protocol, a fluorescence detection system that relies upon a 5′-nuclease assay with allele specific fluorogenic probes. These probes only fluoresce when they are bound to the template. This method is described in Macleod et al. Eur J Clinical Investigation 2001 31(7): 570-3. Commercial products for determining APOE genotype are available from LabCorp and Athena Diagnostics.

Improvement in cognitive function in a patient may be determined by one or more established methods for example ADAS-cog and/or CIBIC+ and/or the DAD method (details of each of which are described elsewhere herein, and the associated references are herein incorporated in their entirety by reference). The preferred method is ADAS-cog. Suitably the improvement in ADAS-cog is at least 1 point, especially at least 2 points over a 24 week treatment period.

Another possible method is the Buschke Selective Reminding Test (Grober E et al. Neurology 1988 38:900-903).

By “improvement in cognitive function” is meant an improvement in cognitive treatment with drug treatment over the passage of time relative to an untreated individual. Since dementia (eg AD) patients typically decline in cognitive function with time an “improvement in cognitive function” embraces a slowing or arrest in decline as well as absolute improvement. As is shown in Example 2, an absolute improvement in cognitive function does appear to result from preferred methods of performing the invention.

The term PPAR-gamma agonist as used herein is meant to include compounds or compositions which behave as agonists or partial agonists of the PPAR-gamma receptor. Suitable PPAR-gamma agonists of use in the present invention include docosahexaenoic acid, prostaglandin J₂, prostaglandin J₂ analogues (e.g. Δ¹²-prostaglandin J₂ and 15-deoxy-Δ^{12,14}-prostaglandin J₂), farglitazar (GI 262570), oxazolidinediones and thiazolidinediones. Exemplary thiazolidinediones include troglitazone, ciglitazone, pioglitazone, rosiglitazone (BRL 49653), darglitazone and englitazone.

Preferably the PPAR-gamma agonist is a thiazolidinedione. More preferably the thiazolidinedione is rosiglitazone or pioglitazone, especially rosiglitazone. Farglitazar is also of particular interest.

Embraced by the present invention are those PPAR-gamma agonists that are selective over other PPAR receptors (e.g. PPAR-alpha and PPAR-delta) (by “selective” it being understood that the agonist activity will be, for example, at least 10 times greater, e.g. at least 50 times greater for PPAR-gamma than for either PPAR-alpha or PPAR-delta). A suitable measure for assessing relative agonist activity is the EC50 value obtained in the Transfection Assay mentioned below. For example a selective PPAR-gamma agonist may have an EC50 value in the PPAR-gamma assay which is at least 10 times lower than the EC50 value obtained for it in either the PPAR-alpha or PPAR-delta assays. Also embraced by the present invention are those PPAR-gamma agonists that also have notable agonist activity against one or more other PPAR receptors e.g. PPAR-alpha and/or PPAR-delta.

PPAR receptor agonist activity may be determined by conventional screening methods. Suitable screens are, for example, those given below:

### Binding Assay:

Compounds may be tested for their ability to bind to hPPAR gamma, hPPAR alpha or hPPAR delta using a Scintillation Proximity Assay (SPA). The PPAR ligand binding domain (LBD) may be expressed in E. coli as polyHis tagged fusion proteins and purified. The LBD may then be labelled with biotin and immobilised on streptavidin-modified scintillation proximity beads. The beads may then be incubated with a constant amount of the appropriate radioligand (5-{4-[2-(Methyl-pyridin-2-yl-amino)-ethoxy]-benzyl}-thiazolidine-2,4-dione (J. Med. Chem. 1994, 37(23), 3977), for PPAR gamma), and labelled GW 2433 (see Brown, P. J et al. Chem. Biol. 1997 4: 909-918), for the structure and synthesis of this ligand) for PPAR alpha and PPAR delta) and variable concentrations of test compound, and after equilibration the radioactivity bound to the beads may be measured by a scintillation counter. The amount of nonspecific binding, as assessed by control wells containing 50 μM of the corresponding unlabeled ligand, is subtracted from each data point. For each compound tested, plots of ligand concentration vs. CPM of radioligand bound may be constructed and apparent Ki values are estimated from nonlinear least squares fit of the data assuming simple competitive binding. The details of this assay have been reported elsewhere (see, Blanchard, S. G. et. al. Anal. Biochem. 1998 257: 112-119).

### Transfection Assay:

Compounds may be screened for functional potency in transient transfection assays in CV-1 cells for their ability to activate the PPAR subtypes (transactivation assay). A previously established chimeric receptor system may be utilized to allow comparison of the relative transcriptional activity of the receptor subtypes on the same target gene and to prevent endogenous receptor activation from complicating the interpretation of results. See, for example, Lehmann, J. M et al J. Biol. Chem., 1995 270:12953-6. The ligand binding domains for murine and human PPAR alpha, PPAR gamma and PPAR delta are each fused to the yeast transcription factor GAL4 DNA binding domain. CV-1 cells are transiently transfected with expression vectors for the respective PPAR chimera along with a reporter construct containing five copies of the GAL4 DNA binding site driving expression of secreted placental alkaline phosphatase (SPAP) and beta-galactosidase. After 16 h, the medium are exchanged to DME medium supplemented with 10% delipidated fetal calf serum and the test compound at the appropriate concentration. After an additional 24 h, cell extracts are prepared and assayed for alkaline phosphatase and beta-galactosidase activity. Alkaline phosphatase activity is corrected for transfection efficiency using the beta-galactosidase activity as an internal standard (see, for example, Kliewer, S. A., et. al. Cell 1995 83: 813-819). Rosiglitazone (BRL 49653) may be used as a positive control in the hPPAR gamma assay. The positive control in the hPPAR alpha assays may be 2-4-[2-(3-[4-fluorophenyl]-1-heptylureido)ethyl]-phenoxy-(2-methyl propionic acid (WO 97/36579). The positive control for PPAR delta assays may be 2-{2-methyl-4-[({4-methyl-2-{trifluoromethyl)phenyl]-1,3-thiazol-5-yl}methyl)sulfanyl]phenoxy}acetic acid (WO 01/00603). An EC50 may be determined as the concentration at which a compound achieves 50% activation relative to the appropriate positive control.

An “agonist” will typically have a pKi of at least 6.0 preferably at least 7.0 to the relevant PPAR in the Binding Assay described above, and achieves at least 50% activation of the relevant PPAR relative to the appropriate indicated positive control in the Transfection Assay described above at concentrations of 10⁻⁵ M or less.

Optionally, more than one PPAR-gamma agonist may be utilised in the present invention (for example, a combination of two PPAR-gamma agonists). In a preferred embodiment of the present invention a single PPAR-gamma agonist is utilised.

The PPAR-gamma agonist according to the present invention will normally be formulated into a pharmaceutical composition in accordance with standard pharmaceutical practice.

It will be clear to those skilled in the art that the medicaments may be presented in the form of pharmaceutically acceptable salts or solvates.

### Suitable Solvates Include Hydrates.

Suitable salts include those formed with both organic and inorganic acids or bases.

Pharmaceutically acceptable acid addition salts include those formed from hydrochloric, hydrobromic, sulphuric, citric, tartaric, phosphoric, lactic, pyruvic, acetic, trifluoroacetic, triphenylacetic, sulphamic, sulphanilic, succinic, oxalic, fumaric, maleic, malic, glutamic, aspartic, oxaloacetic, methanesulphonic, ethanesulphonic, arylsulphonic (for example p-toluenesulphonic, benzenesulphonic, naphthalenesulphonic or naphthalenedisulphonic), salicylic, glutaric, gluconic, tricarballylic, cinnamic, substituted cinnamic (for example, phenyl, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4 methoxybenzoic, 2- or 4-hydroxybenzoic, 4-chlorobenzoic, 4-phenylbenzoic, benzeneacrylic (for example 1,4-benzenediacrylic) and isethionic acids.

Pharmaceutically acceptable base salts include ammonium salts, alkali metal salts such as those of sodium and potassium, alkaline earth metal salts such as those of calcium and magnesium and salts with organic bases such as dicyclohexylamine and N-methyl-D-glucamine.

Where the PPAR-gamma agonist is rosiglitazone, it is preferred that the rosiglitazone is in the form of rosiglitazone maleate. Where the PPAR-gamma agonist is pioglitazone, it is preferred that the pioglitazone is in the form of pioglitazone hydrochloride. Where the PPAR-gamma agonist is farglitazar, an exemplary salt form is the sodium salt.

Suitable formulations include those for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration, although the most suitable route may depend upon for example the condition of the recipient and the medicament in question. The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of use in the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di- or poly-saccharides (e.g. lactose or starch). Use of lactose is preferred.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants e.g. oleic acid or lecithin and cosolvents e.g. ethanol. Pressurised formulations will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 um, preferably 2-5 um. Particles having a size above 20 um are generally too large when inhaled to reach the small airways. To achieve these particle sizes the particles of the active ingredient as produced may be size reduced by conventional means e.g. by micronisation. The desired fraction may be separated out by air classification or sieving. Preferably, the particles will be crystalline. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 um and not less than 15% will have a MMD of less than 15 um.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose and acacia.

It should be understood that in addition to the ingredients particularly mentioned above, the formulations of this invention may include other agents conventional in the art having regard to the type of formulation in question, for example those suitable for oral administration may include flavouring agents.

Where the PPAR-gamma agonist is rosiglitazone or pioglitazone, the compounds are preferably formulated for oral administration, in particular as a tablet.

In light of the cognitive issues associated with MCI, AD or other dementias it may be desirable that the PPAR-gamma agonist is formulated for sustained release, thereby reducing the required frequency of administration (for example to a single daily dose).

When the PPAR-gamma agonist is rosiglitazone, extended release formulations eg of the type disclosed in WO05/013935 are particularly suitable (although these formulations can also be applied to other PPAR-gamma agonists). The tablets described therein are comprised of a core which contains two different active compositions, an immediate release formulation and a modified release formulation. Furthermore, the tablet is surrounded by a coating of hydroxypropyl methylcellulose (HPMC) though which two holes penetrate, one to the immediate release depot and one to the modified release depot. The arrangement ensures a highly controlled dissolution of the rosiglitazone. A single tablet of 2 mg, 4 mg or 8 mg (e.g. 8 mg) may for example be administered once per day.

Thus there is provided as an aspect of the invention a method, PPAR-gamma agonist, use or kit as previously described wherein the PPAR-gamma agonist is presented as an extended release tablet comprising a core which contains a depot of an immediate release formulation and a depot of a modified release formulation. In particular there is provided a method, PPAR-gamma agonist, use or kit wherein said tablet is surrounded by a coating eg of HPMC through which holes penetrate; at least one (eg one) penetrating to the immediate release depot and at least one (eg one) penetrating to the modified release depot.

A rosiglitazone 8 mg extended release tablet of this sort may typically contain 3 mg of rosiglitazone within the immediate release depot and 5 mg of rosiglitazone within the modified release depot. A rosiglitazone 4 mg extended release tablet of this sort may typically contain 1.5 mg of rosiglitazone within the immediate release depot and 2.5 mg of rosiglitazone within the modified release depot. A rosiglitazone 2 mg extended release tablet of this sort may typically contain 0.75 mg of rosiglitazone within the immediate release depot and 1.25 mg of rosiglitazone within the modified release depot.

Suitable daily doses of PPAR-gamma agonist will be apparent to those skilled in the art and will depend upon the particular PPAR-gamma agonist which has been chosen. For example, in the case of rosiglitazone, the daily dose will typically be in the range 0.01 mg to 12 mg (for example 2 mg, 4 mg or 8 mg daily). A daily dose of 8 mg or more eg 8 mg may be especially suitable.

In the context of the application of the present invention to APOE4 heterozygotes, administration of higher doses of rosiglitazone (eg 4 mg or more, for example 4 mg or 8 mg) would seem to be advantageous.

The PPAR-gamma agonist of use in the present invention may be administered in combination with one or more further medicaments of use for the treatment or prevention of Alzheimer's disease. Further medicaments for the treatment or prevention of Alzheimer's disease include cholinesterase inhibitors (for example tacrine, galantamine, rivastigamine or donepezil) and NMDA inhibitors (for example memantine). The PPAR-gamma agonist of use in the present invention may be administered in combination with one or more further medicaments of use for the treatment or prevention of other dementias. Other further medicaments include non-steroidal anti-inflammatory drugs (NSAIDs) such as such as naproxen, ibuprofen, diclofenac, indomethacin, nabumetone, piroxicam, celecoxib and aspirin. Other medicaments that may be combined with the PPAR-gamma agonists in the present invention include HMG-CoA reductase inhibitors such as statins (eg simvastatin (Zocor), atovastatin (Lipitor), rosuvastatin (Crestor), fluvastatin (Lescol)).

Combination of the PPAR-gamma agonist of use in the present invention (particularly rosiglitazone, eg rosiglitazone maleate) with donepezil (eg donepezil hydrochloride) may be of particular interest.

Depending on the individual medicaments utilised in a combination therapy for simultaneous administration, they may be formulated in combination (where a stable formulation may be prepared and where desired dosage regimes are compatible) or the medicaments may be formulated separately (for concomitant or separate administration through the same or alternative routes).

It will be understood that the methods and uses of the invention may be employed prophylaxis as well as (more suitably) in the treatment of subjects suffering from mild cognitive impairment, Alzheimer's disease or other dementias.

The term simultaneous administration as used herein in relation to the administration of medicaments refers to the administration of medicaments such that the individual medicaments are present within a subject at the same time. In addition to the concomitant administration of medicaments (via the same or alternative routes), simultaneous administration may include the administration of the medicaments (via the same or an alternative route) at different times.

### EXAMPLES

### Example 1

### Preparation of Rosiglitazone Maleate Extended Release Tablets

Extended release tablets containing 2 mg, 4 mg or 8 mg of the PPAR-gamma agonist rosiglitazone (in the form of the maleate salt) were prepared according to the methods described in WO05/013935 (corresponding to Example 3 therein).

(a) 2 mg rosiglitazone extended release tablet

A core was formed from the following compositions:

TABLE 2
2 mg rosiglitazone tablet first composition
(immediate release layer).

| Component | Proportion (% w/w) |
|---|---|
| Rosiglitazone (as maleate salt) | 1.99 |
| Lactose | 97.48 |
| Yellow iron oxide | 0.03 |
| Magnesium stearate | 0.5 |

TABLE 3
2 mg rosiglitazone tablet second composition
(modified release layer).

| Component | Proportion (% w/w) |
|---|---|
| Rosiglitazone (as maleate salt) | 1.1 |
| HPMC | 30.0 |
| Lactose | 66.9 |
| Silicon dioxide | 0.5 |
| Magnesium stearate | 1.5 |

by compression to form 7 mm normal concave bilayer tablets of 200 mg (50 mg of the immediate release layer and 150 mg of the modified release layer).

The tablet cores were coated with a HPMC-based sub-coat and a polymethacrylate resin soluble at pH 5.5 to a total weight of 217.3 mg.

An opening of diameter 3.0 mm was drilled through the coating in each of the two primary surfaces of the coated cores to expose the surface of the core.

The final tablet contained 2 mg rosiglitazone −0.75 mg rosiglitazone within the immediate release layer and 1.25 mg rosiglitazone within the modified release layer.

(b) 4 mg rosiglitazone extended release tablet

A core was formed from the following compositions:

TABLE 4
4 mg rosiglitazone tablet first composition
(immediate release layer).

| Component | Proportion (% w/w) |
|---|---|
| Rosiglitazone (as maleate salt) | 3.98 |
| Lactose | 95.49 |
| Yellow iron oxide | 0.03 |
| Magnesium stearate | 0.5 |

TABLE 5
4 mg rosiglitazone tablet second composition
(modified release layer).

| Component | Proportion (% w/w) |
|---|---|
| Rosiglitazone (as maleate salt) | 2.2 |
| HPMC | 30.0 |
| Lactose | 65.8 |
| Silicon dioxide | 0.5 |
| Magnesium stearate | 1.5 |

by compression to form 7 mm normal concave bilayer tablets of 200 mg (50 mg of the immediate release layer and 150 mg of the modified release layer).

The tablet cores were coated with a HPMC-based sub-coat and a polymethacrylate resin soluble at pH 5.5 to a total weight of 217.3 mg.

An opening of diameter 3.0 mm was drilled through the coating in each of the two primary surfaces of the coated cores to expose the surface of the core.

The final tablet contained 4 mg rosiglitazone −1.5 mg rosiglitazone within the immediate release layer and 2.5 mg rosiglitazone within the modified release layer.

(a) 8 mg rosiglitazone maleate extended release tablet

A core was formed from the following compositions:

TABLE 6
8 mg rosiglitazone tablet first composition
(immediate release layer).

| Component | Proportion (% w/w) |
|---|---|
| Rosiglitazone (as maleate salt) | 7.95 |
| Lactose | 91.52 |
| Yellow iron oxide | 0.03 |
| Magnesium stearate | 0.5 |

TABLE 7
8 mg rosiglitazone tablet second composition
(modified release layer).

| Component | Proportion (% w/w) |
|---|---|
| Rosiglitazone (as maleate salt) | 4.4 |
| HPMC | 30.0 |
| Lactose | 63.6 |
| Silicon dioxide | 0.5 |
| Magnesium stearate | 1.5 |

by compression to form 7 mm normal concave bilayer tablets of 200 mg (50 mg of the immediate release layer and 150 mg of the modified release layer).

The tablet cores were coated with a HPMC-based sub-coat and a polymethacrylate resin soluble at pH 5.5 to a total weight of 217.3 mg.

An opening of diameter 3.0 mm was drilled through the coating in each of the two primary surfaces of the coated cores to expose the surface of the core.

The final tablet contained 8 mg rosiglitazone −3 mg rosiglitazone within the immediate release layer and 5 mg rosiglitazone within the modified release layer.

### Example 2

### The Effect of PPAR-gamma agonist (rosiglitazone maleate) Treatment on ADAS-Cog and CIBIC+ in Alzheimer's Patients

### Method

Analysis for the full intent to treat (ITT) population was performed on 511 subjects who were randomly allocated into one of four specific treatment regimes. Genotyping analysis was performed on 63% (323/511) of the ITT population.

Patient population included Caucasian males and females between 50-85 years of age who had been diagnosed with mild to moderate AD, were not receiving any medications which could adversely prejudice the study (e.g. PPAR-gamma agonists or conventional AD medicaments) or had any other potentially prejudicial ailments (e.g. diabetes or major psychiatric disorders).

Patients received either placebo or one of three dosage levels of extended release rosiglitazone provided once daily (2 mg, 4 mg and 8 mg tablets as described in Example 1). Patients were examined using the cognitive Alzheimer's Disease Assessment Scale (ADAS-cog; for further information see Rosen W G et al. Am. J. Psychiatry. 1984 141:1356-1364) and the Clinician's Interview-Based Impression of Change with caregiver information (CIBIC+; for further information see Knopman D S et al. Neurology 1994 44: 2315-2321); and secondary assessments were performed using: the Disability Assessment for Dementia (DAD, for further information see Gelinas L et al. Am J Occup Ther 1999 53: 471-81) and the Neuropsychiatric Inventory test (NPI, for further information see Cummings et al (1994) Neurology 44, 2308-2314) at the start of the study (baseline) and during the course of the study (after 8, 16 and 24 weeks of treatment).

APOE genotype was determined using the TaqMan PCR-based method of McLeod et al 2001 infra.

All statistics reflect last observed assessment carried forward (LOCF) measurements.

Tables 8 and 9 summarise the age and sex details of the genotyped and full ITT populations by treatment regime.

TABLE 8
Summary of genotyped population.

| | | | Rosiglitazone | Rosiglitazone | Rosiglitazone | |
|---|---|---|---|---|---|---|
| | | Placebo | 2 mg | 4 mg | 8 mg | Total |
| | | N = 78 | N = 85 | N = 80 | N = 80 | N = 323 |
| Age | Mean | 71.2 | 70 | 68.8 | 70.5 | 70.1 |
| | (SD) | (8.94) | (8.58) | (9.56) | (8.02) | (8.79) |
| Sex | Female | 51 | 53 | 47 | 54 | 205 |
| | | (65%) | (62%) | (59%) | (68%) | (63%) |
| | Male | 27 | 32 | 33 | 26 | 118 |
| | | (35%) | (38%) | (41%) | (33%) | (37%) |

TABLE 9
Summary of full intent to treat population.

| | | | Rosiglitazone | Rosiglitazone | Rosiglitazone | |
|---|---|---|---|---|---|---|
| | | Placebo | 2 mg | 4 mg | 8 mg | Total |
| | | N = 122 | N = 127 | N = 130 | N = 132 | N = 511 |
| Age | Mean | 71.8 | 70.9 | 69.7 | 70.5 | 70.7 |
| | (SD) | (8.23) | (8.46) | (8.97) | (8.47) | (8.55) |
| Sex | Female | 77 | 71 | 73 | 87 | 308 |
| | | (63%) | (56%) | (56%) | (66%) | (60%) |
| | Male | 45 | 56 | 57 | 45 | 203 |
| | | (37%) | (44%) | (44%) | (34%) | (40%) |

### Results

It should be noted that in the case of ADAS-cog higher scores indicate reduced cognitive function. A negative change from baseline over the course of the study therefore shows an improvement and a positive change from baseline shows decline. Similarly a negative treatment difference shows that treatment resulted in improvement relative to placebo and a positive treatment difference shows that treatment resulted in decline relative to placebo.

Higher CIBIC+ scores indicate a greater level of decline with scores below 4 denoting clinical improvement and scores above 4 denoting clinical decline. A negative CIBIC+ treatment difference therefore shows that treatment resulted in an improvement relative to placebo and a positive treatment difference shows that treatment resulted in decline relative to placebo.

### (i) ITT Population

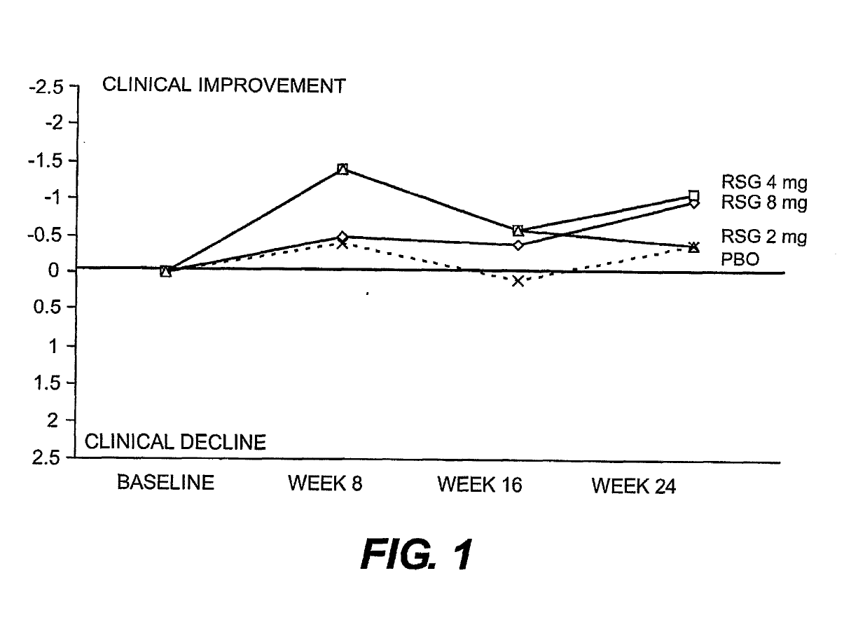

Table 10 summarises the model adjusted change in ADAS-cog from baseline and CIBIC+ results at the end of the 24 week trial for each of the four treatment regimes in the ITT population. FIG. 1 shows the model adjusted ADAS-cog change from baseline in the ITT population during the course of the study (the analyses included adjustments for effects of baseline score, country, mini mental state examination screening and baseline body mass index).

The ADAS-cog data in Table 10 and FIG. 1 support a trend of clinical improvement (i.e. a negative change from baseline) as a result of treatment using the PPAR-gamma agonist rosiglitazone. At all time points there is a net improvement in the analysed population as a whole. However statistical analysis of the effect of rosiglitazone treatment on AD patients indicates that this trend is not statistically significant. The CIBIC+ results did not lead to a distinguishable difference between treatment groups and placebo at 24 weeks.

TABLE 10
Summary of model adjusted ADAS-cog change from baseline
after 24 weeks by treatment group (LOCF-ITT population).

| | | | Treatment | P-value |
|---|---|---|---|---|
| | | | Difference (95% | for |
| | Treatment | LSMean | confidence limits) | Treatment |
| Variable | Regime | (SE) | Rosi-Placebo | Difference |
| ADAS-cog | Placebo | −0.4 (0.55) | | |
| | (n = 122) | | | |
| | Rosi 2 mg | −0.2 (0.54) | 0.25 (−1.19, 1.68) | 0.74 |
| | (n = 126) | | | |
| | Rosi 4 mg | −0.9 (0.54) | −0.46 (−1.90, 0.97) | 0.52 |
| | (n = 128) | | | |
| | Rosi 8 mg | −0.7 (0.53) | −0.27 (−1.70, 1.16) | 0.71 |
| | (n = 130) | | | |
| CIBIC+ | Placebo | 4.0 (0.10) | | |
| | (n = 122) | | | |
| | Rosi 2 mg | 3.8 (0.10) | −0.16 (−0.44, 0.11) | 0.23 |
| | (n = 126) | | | |
| | Rosi 4 mg | 3.8 (0.10) | −0.16 (−0.43, 0.11) | 0.24 |
| | (n = 128) | | | |
| | Rosi 8 mg | 3.8 (0.10) | −0.22 (−0.49, 0.05) | 0.11 |
| | (n = 130) | | | |

### (ii) Genotyped Population

Table 11 and Table 11a (which reflects the inclusion of 2 additional subjects) indicate the results of APOE4 allele determination in the genotypes population. Treatment regimes were allocated prior to APOE4 allele determination, despite this, there is generally a good distribution of phenotypes between the various groupings as a result of statistical averaging, although some of the less prevalent phenotypes show some clustering (for example a large proportion of the APOE4 homozygotes are in the 8 mg rosiglitazone treatment group).

TABLE 11
Summary of APOE allele status by treatment group.

| | | | Rosi | Rosi | Rosi | |
|---|---|---|---|---|---|---|
| | | Placebo | 2 mg | 4 mg | 8 mg | Total |
| | | N = 78 | N = 85 | N = 80 | N = 80 | N = 323 |
| APOE | N | 78 | 85 | 79 | 78 | 320 |
| Genotype | | (100%) | (100%) | (100%) | (100%) | (100%) |
| | 4.4 | 5 | 4 | 6 | 12 | 27 |
| | | (6%) | (5%) | (8%) | (15%) | (8%) |
| | 3.4 | 27 | 31 | 27 | 22 | 107 |
| | | (35%) | (37%) | (34%) | (28%) | (33%) |
| | 2.4 | 3 | 1 | 1 | 2 | 7 |
| | | (4%) | (1%) | (1%) | (3%) | (2%) |
| | 3.3 | 35 | 43 | 37 | 35 | 150 |
| | | (45%) | (51%) | (47%) | (45%) | (47%) |
| | 2.3 | 8 | 6 | 7 | 7 | 28 |
| | | (10%) | (7%) | (9%) | (9%) | (9%) |
| | 2.2 | 0 | 0 | 1 | 0 | 1 |
| | | | | (1%) | | (<1%) |
| APOE4 | 2 | 5 | 4 | 6 | 12 | 27 |
| Copies | | (6%) | (5%) | (8%) | (15%) | (8%) |
| | 1 | 30 | 32 | 28 | 24 | 114 |
| | | (38%) | (38%) | (35%) | (31 %) | (36%) |
| | 0 | 43 | 49 | 45 | 42 | 179 |
| | | (55%) | (58%) | (57%) | (54%) | (56%) |
| APOE4 | Yes | 35 | 36 | 34 | 36 | 141 |
| Carriage | | (45%) | (42%) | (43%) | (46%) | (44%) |
| | No | 43 | 49 | 45 | 42 | 179 |
| | | (55%) | (58%) | (57%) | (54%) | (56%) |

TABLE 11a
Summary of APOE allele status by treatment group.

| | | | Rosi | Rosi | Rosi | |
|---|---|---|---|---|---|---|
| | | Placebo | 2 mg | 4 mg | 8 mg | Total |
| | | N = 78 | N = 85 | N = 80 | N = 80 | N= 323 |
| APOE | N | 78 | 85 | 80 | 79* | 322 |
| Genotype | | (100%) | (100%) | (100%) | (100%) | (100%) |
| | 4.4 | 5 | 4 | 6 | 12 | 27 |
| | | (6%) | (5%) | (8%) | (15%) | (8%) |
| | 3.4 | 27 | 31 | 28 | 22 | 108 |
| | | (35%) | (37%) | (35%) | (28%) | (34%) |
| | 2.4 | 3 | 1 | 1 | 2 | 7 |
| | | (4%) | (1%) | (1%) | (3%) | (2%) |
| | 3.3 | 35 | 43 | 37 | 36 | 151 |
| | | (45%) | (51%) | (46%) | (46%) | (47%) |
| | 2.3 | 8 | 6 | 7 | 7 | 28 |
| | | (10%) | (7%) | (9%) | (9%) | (9%) |
| | 2.2 | 0 | 0 | 1 | 0 | 1 |
| | | | | (1%) | | (<1%) |
| APOE4 | 2 | 5 | 4 | 6 | 12 | 27 |
| Copies | | (6%) | (5%) | (8%) | (15%) | (8%) |
| | 1 | 30 | 32 | 29 | 24 | 115 |
| | | (38%) | (38%) | (36%) | (30%) | (36%) |
| | 0 | 43 | 49 | 45 | 43 | 180 |
| | | (55%) | (58%) | (56%) | (54%) | (56%) |
| APOE4 | Yes | 35 | 36 | 35 | 36 | 142 |
| Carriage | | (45%) | (42%) | (44%) | (46%) | (44%) |
| | No | 43 | 49 | 45 | 43 | 180 |
| | | (55%) | (58%) | (56%) | (54%) | (56%) |

*no genotype information available for one subject

A breakdown of the change in ADAS-cog at the end of the 24 week study by APOE allele status and treatment regime is shown in Table 12 below.

A prospectively defined test for interaction between APOE carriage status and ADAS-cog total score change from baseline to week 24 was significant (P=0.0194). Subsequent exploratory testing revealed that APOE4− (those without an APOE4 allele) patients, after 24 weeks, showed a general trend of improvement in cognitive function as a result of treatment with the PPAR-gamma agonist rosiglitazone, there being evidence that this improvement was due to treatment at the highest 8 mg rosiglitazone dosage compared to placebo (P=0.027).

APOE4 heterozygotes (those with a single APOE4 allele) do not show any recognisable trend. Although there is some decline in the group receiving 2 mg rosiglitazone, both the 4 mg and 8 mg dose regimes show little change, and none of the points are individually significant after 24 weeks of treatment.

APOE4 homozygotes (those with two APOE4 alleles) show a relatively large positive change in ADAS-cog scores as a result of rosiglitazone treatment. There was some evidence that this decline was due to treatment at all three dosage levels after 24 weeks of treatment (unadjusted P<0.05), although sample numbers are small. However, the extent of clinical decline as a result of treatment decreases with increasing dosage level. It is not clear whether the clinical decline in the treated group is due to rosiglitazone or due to the natural progression of Alzheimer's disease.

TABLE 12
Summary of model-adjusted ADAS-cog change from baseline
after 24 weeks by APOE4 allele status and treatment group
(PGx ITT population).

| | | | Treatment | |
|---|---|---|---|---|
| | | | Difference | P-value |
| | | | (90% confidence | for |
| APOE4 | Treatment | LSMean | limits) | Treatment |
| Copies | Regime | (SE) | Rosi-Placebo | Difference |
| 0 | Placebo | 1.01 (0.95) | | |
| | (n = 43) | | | |
| | Rosi 2 mg | −1.38 (0.90) | −2.39 (−4.43, −0.36) | 0.053 |
| | (n = 49) | | | |
| | Rosi 4 mg | −1.25 (0.90) | −2.26 (−4.32, −0.20) | 0.071 |
| | (n = 45) | | | |
| | Rosi 8 mg | −1.85 (0.94) | −2.86 (−4.98, −0.74) | 0.027 |
| | (n = 42) | | | |
| 1 | Placebo | −0.57 (1.10) | | |
| | (n = 30) | | | |
| | Rosi 2 mg | 2.02 (1.10) | 2.59 (0.10, 5.08) | 0.087 |
| | (n = 32) | | | |
| | Rosi 4 mg | −0.21 (1.15) | 0.36 (−2.18, 2.90) | 0.82 |
| | (n = 28) | | | |
| | Rosi 8 mg | −0.34 (1.25) | 0.23 (−2.42, 2.87) | 0.89 |
| | (n = 24) | | | |
| 2 | Placebo | −4.58 (2.70) | | |
| | (n = 5) | | | |
| | Rosi 2 mg | 5.67 (2.98) | 10.26 (3.64, 16.87) | 0.011 |
| | (n = 4) | | | |
| | Rosi 4 mg | 3.16 (2.44) | 7.75 (1.79, 13.71) | 0.033 |
| | (n = 6) | | | |
| | Rosi 8 mg | 1.91 (1.73) | 6.50 (1.28, 11.71) | 0.041 |
| | (n = 12) | | | |

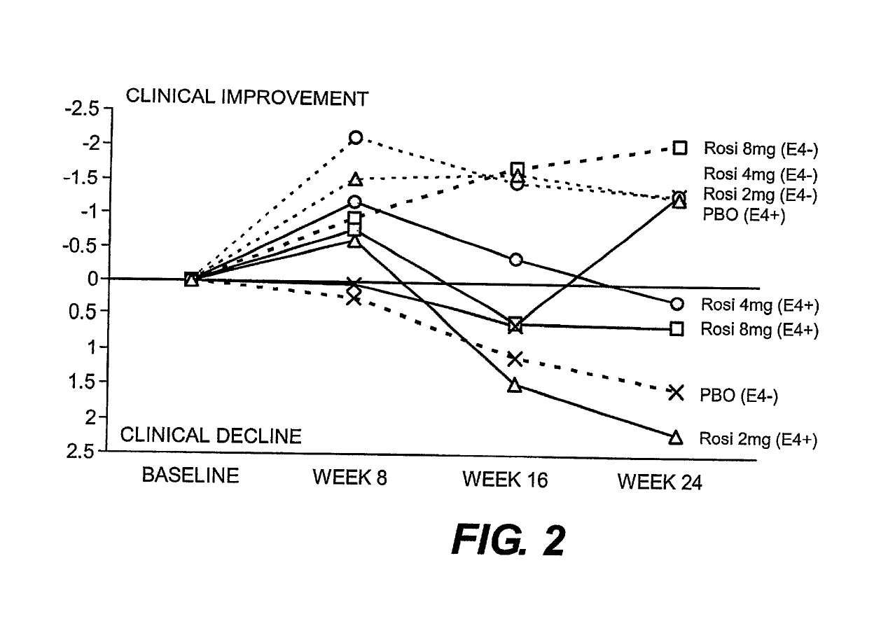

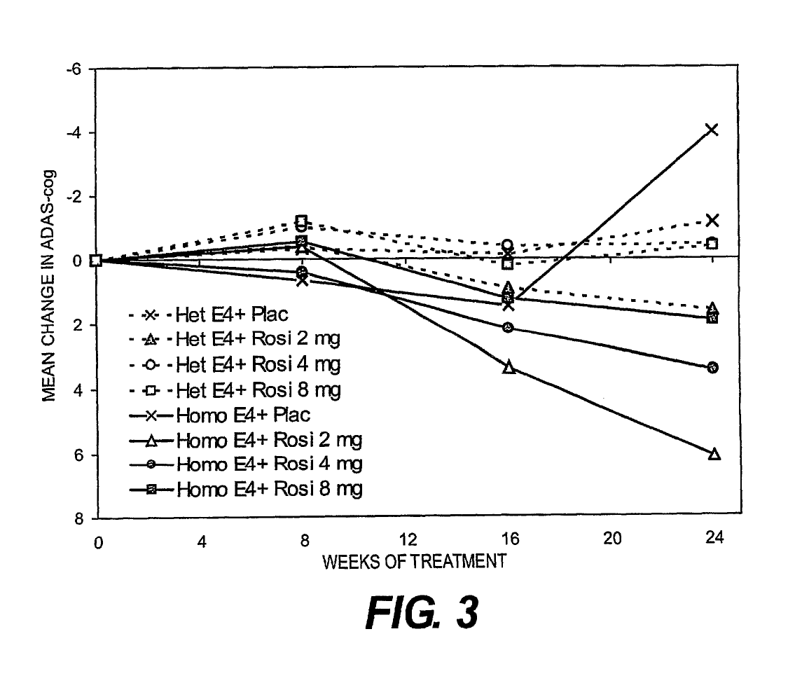

FIG. 2 shows a plot of the model adjusted ADAS-cog change from baseline in the analysed population by treatment regime and APOE allele status (carriers of 1 or 2 APOE4 alleles being shown together). FIG. 3 shows a plot in which data on the APOE4 heterozygotes (indicated by ‘Het E4+’) have been separated from data on the APOE4 homozygotes (indicated by ‘Homo E4+’).

A clear trend of cognitive improvement as a result of rosiglitazone treatment is particularly apparent in the APOE4− individuals. At all time points (8, 16 and 24 weeks) the placebo group shows a continued decline in cognitive function, whereas those treated with 2 mg, 4 mg or 8 mg of the PPAR-gamma agonist show marked improvement.

The situation with respect to APOE4+ individuals is less clear. After 8 weeks of treatment, those receiving placebo show a slight decline in cognitive function, while all those receiving rosiglitazone (2 mg, 4 mg or 8 mg) slow slight improvement. After 16 weeks of treatment those receiving placebo show a continued decline in cognitive function, although treatment with 4 mg and 8 mg shows the same or better clinical status. Treatment with 2 mg rosiglitazone shows a greater decline than the placebo. Finally, after 24 weeks of treatment, a large and surprising improvement in APOE4 carriers receiving placebo is observed. This apparent improvement may have been influenced by a small number of subjects with unexpected and large improvements in ADAS-cog scores. All three rosiglitazone treatment arms finish with a clinical decline, and as a result of the unusual improvement in the placebo arm at this time point, rosiglitazone treatment appears to depict a clinical decline compared to the placebo. It is possible that the clinical decline observed in some APOE4+ groups is due to the natural clinical course of AD.

FIG. 3 shows the results for the APOE4 heterozygotes separated from those for the APOE4 homozygotes. Although the number of APOE4 homozygotes is small, it can be seen that whereas all APOE4 homozygotes treated with rosiglitazone experienced a clinical decline, the APOE4 heterozygotes who received the higher doses of rosiglitazone (4, 8 mg) remained close to the baseline for the course of the study.

Similar results were identified using the Disability Assessment for Dementia (DAD) test (Gelinas L et al. Am J Occup Ther 1999 53: 471-81). A prospectively defined test for interaction between APOE4 carriage status and DAD scores at week 24 was significant (P=0.006). Subsequent testing demonstrated a pattern of results that is qualitatively similar to that for ADAS-Cog: namely, APOE4− subjects demonstrated improvement on DAD, whereas APOE4+ subjects demonstrated no improvement.

Similar results were identified using the Neuropsychiatric Inventory (NPI) test (Cummings et al (1994) Neurology 44, 2308-2314). A prospectively defined test for interaction between APOE4 carriage status and NPI scores at week 24 was significant (P=0.086). Subsequent testing demonstrated a pattern of results that is qualitatively similar to that for ADAS-Cog: namely, APOE4− subjects demonstrated improvement on NPI, whereas APOE4+subjects demonstrated no improvement.

Table 13 and Table 13a (which is an updated analysis taking into account additional subjects) shows the CIBIC+results after 24 weeks, separated by APOE4 allele status and treatment regime. There was no evidence of an interaction between treatment and APOE4 copies, so the differences described below between the subgroups are likely to be due to random error rather than any differential effect.

APOE4− (those without an APOE4 allele) patients all show slight improvement over the 24 week period, with the greatest improvement observed in the group treated with 2 mg of rosiglitazone (unadjusted P=0.052).

APOE4 heterozygotes (those with a single APOE4 allele) show a decline in the group treated with 2 mg of rosiglitazone (P=0.056). Less decline is shown in the group receiving 4 mg of rosiglitazone and a slight improvement is seen in the group receiving 8 mg rosiglitazone (although no comparison came close to significance in the exploratory analysis).

APOE4 homozygotes (those with two APOE4 alleles) all slow slight improvement in the CIBIC+upon treatment for 24 weeks compared to placebo, although the extent of improvement decreased with treatment dosage.

TABLE 13
Summary of model adjusted CIBIC+ after 24 weeks by APOE4
allele status and treatment group (PGx ITT population).

| | | | Treatment | |
|---|---|---|---|---|
| | | | Difference | P-value |
| | | | (90% confidence | for |
| APOE4 | Treatment | LSMean | limits) | Treatment |
| Copies | Regime | (SE) | Rosi-Placebo | Difference |
| 0 | Placebo | 3.95 (0.19) | | |
| | (n = 41) | | | |
| | Rosi 2 mg | 3.47 (0.18) | −0.49 (−0.89, −0.08) | 0.051 |
| | (n = 45) | | | |
| | Rosi 4 mg | 3.76 (0.18) | −0.19 (−0.60, 0.22) | 0.44 |
| | (n = 43) | | | |
| | Rosi 8 mg | 3.76 (0.18) | −0.19 (−0.61, 0.22) | 0.44 |
| | (n = 42) | | | |
| 1 | Placebo | 3.88 (0.22) | | |
| | (n = 28) | | | |
| | Rosi 2 mg | 4.47 (0.23) | 0.59 (0.08, 1.11) | 0.056 |
| | (n = 28) | | | |
| | Rosi 4 mg | 4.11 (0.23) | 0.23 (−0.28, 0.74) | 0.45 |
| | (n = 25) | | | |
| | Rosi 8 mg | 3.68 (0.25) | −0.20 (−0.73, 0.34) | 0.54 |
| | (n = 23) | | | |
| 2 | Placebo | 4.34 (0.53) | | |
| | (n = 5) | | | |
| | Rosi 2 mg | 3.42 (0.58) | −0.92 (−2.20, 0.37) | 0.24 |
| | (n = 4) | | | |
| | Rosi 4 mg | 3.95 (0.47) | −0.39 (−1.55, 0.77) | 0.58 |
| | (n = 6) | | | |
| | Rosi 8 mg | 4.27 (0.34) | −0.07 (−1.08, 0.95) | 0.91 |
| | (n = 12) | | | |

TABLE 13a
Summary of model adjusted CIBIC+ after 24 weeks by APOE4
allele status and treatment group (PGx ITT population).

| | | | Treatment | |
|---|---|---|---|---|
| | | | Difference | P-value |
| | | | (90% confidence | for |
| APOE4 | Treatment | LSMean | limits) | Treatment |
| Copies | Regime | (SE) | Rosi-Placebo | Difference |
| 0 | Placebo | 3.97 (0.18) | | |
| | (n = 43) | | | |
| | Rosi 2 mg | 3.51 (0.17) | −0.46 (−0.85, −0.07) | 0.052 |
| | (n = 49) | | | |
| | Rosi 4 mg | 3.75 (0.17) | −0.22 (−0.62, 0.18) | 0.37 |
| | (n = 44) | | | |
| | Rosi 8 mg | 3.75 (0.18) | −0.22 (−0.62, 0.19) | 0.38 |
| | (n = 43) | | | |
| 1 | Placebo | 3.92 (0.21) | | |
| | (n = 30) | | | |
| | Rosi 2 mg | 4.32 (0.21) | 0.39 (−0.09, 0.87) | 0.18 |
| | (n = 31) | | | |
| | Rosi 4 mg | 3.97 (0.22) | 0.04 (−0.44, 0.53) | 0.88 |
| | (n = 29) | | | |
| | Rosi 8 mg | 3.70 (0.24) | −0.22 (−0.74, 0.29) | 0.48 |
| | (n = 23) | | | |
| 2 | Placebo | 4.38 (0.52) | | |
| | (n = 5) | | | |
| | Rosi 2 mg | 3.46 (0.57) | −0.91 (−2.19, 0.36) | 0.24 |
| | (n = 4) | | | |
| | Rosi 4 mg | 3.93 (0.47) | −0.45 (−1.59, 0.70) | 0.52 |
| | (n = 6) | | | |
| | Rosi 8 mg | 4.29 (0.33) | −0.09 (−1.09, 0.92) | 0.89 |
| | (n = 12) | | | |

APOE4 copies *treatment interaction P-value = 0.21

### Discussion

The results of Example 2 show that treatment of AD patients using the PPAR-gamma agonist rosiglitazone leads to a non-statistically significant trend to a general improvement in the ITT population as a whole.

In the population tested, there was evidence of a cognitive improvement (as measured in ADAS-cog) in patients without the APOE4 allele on 8 mg rosiglitazone. The mean change from placebo over 24 weeks in patients without the APOE4 allele on 8 mg rosiglitazone was −2.86, P=0.027.

In the population tested, there was no evidence of on-treatment cognitive improvement (as measured by ADAS-cog) in patients carrying the APOE4 allele. However separation of patients carrying one copy from those carrying two copies of the APOE4 allele suggests greatest cognitive decline (as measured by ADAS-cog) in patients carrying two copies of the APOE4 allele (which may, however, be due to the natural progression of the disease rather than response to rosiglitazone) with no notable trend (eg possible stabilisation of cognitive function) in patients carrying one copy of the APOE4 allele.

All references referred to in this application, including patents and patent applications, are incorporated herein by reference to the fullest extent possible. Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

1. A method for improving cognitive function in a subject suffering from or susceptible to MCI, Alzheimer's disease or other dementias, which subject is not homozygous for the APOE4 allele, comprising the steps of:
(i) screening the subject to determine that the subject is not homozygous for the APOE4 allele; and then
(ii) administering a safe and effective amount of a PPAR-gamma agonist to said subject.

2. A method according to claim 1 wherein screening step (i) involves determining that the subject is APOE4−.

3. A method according to claim 1 wherein screening step (i) involves determining that the subject carries a single copy of the APOE4 allele.

4. A method of screening a subject suffering from or susceptible to MCI, Alzheimer's disease or other dementias as an aid in predicting the subject's response to administration of a PPAR-gamma agonist, comprising screening to determine whether the subject carries zero or one copy of the APOE4 allele.

5. A method according to claim 4 wherein the screening involves screening to determine whether the subject is APOE4−.

6. A method according to claim 4 wherein the screening involves screening to determine whether the subject carries a single copy of the APOE4 allele.

7. A method of improving cognitive function in a subject suffering from or susceptible to MCI, Alzheimer's disease or other dementias, which subject has been predetermined not to be homozygous for the APOE4 allele, which method comprises administering a safe and effective amount of a PPAR-gamma agonist to said subject.

8. A PPAR-gamma agonist for use in improving cognitive function in a subject suffering from or susceptible to MCI, Alzheimer's disease or other dementias, which subject has been pre-determined not to be homozygous for the APOE4 allele.

9-10. (canceled)

11. A method, PPAR-gamma agonist or use according to claim 7 wherein the subject has been pre-determined to be APOE4−.

12. A method, PPAR-gamma agonist or use according to claim 7 wherein the subject has been pre-determined to carry a single copy of the APOE4 allele.

13. A method of improving cognitive function in a subject suffering from or susceptible to MCI, Alzheimer's disease or other dementias, which subject is not homozygous for the APOE4 allele, which method comprises administering a safe and effective amount of a PPAR-gamma agonist to said subject.

14. A PPAR-gamma agonist for use in improving cognitive function in a subject suffering from or susceptible to MCI, Alzheimer's disease or other dementias, which subject is not homozygous for the APOE4 allele.

15-16. (canceled)

17. A method, PPAR-gamma agonist or use according to claim 13 wherein the subject is APOE4−.

18. A method, PPAR-gamma agonist or use according to claim 13 wherein the subject carries a single copy of the APOE4 allele.

19. A kit comprising (i) a PPAR-gamma agonist and (ii) instructions directing administration of the PPAR gamma agonist to a subject who is not homozygous for the APOE4 allele.

20. A kit according to claim 19 wherein the instructions direct administration of the PPAR gamma agonist to a subject suffering from or susceptible to MCI, Alzheimer's disease or other dementias who is not homozygous for the APOE4 allele.

21. A kit according to claim 19 wherein the subject has been pre-determined not to be homozygous for the APOE4 allele.

22. A kit according to claim 19 wherein the subject is APOE4−.

23. A kit according to claim 19 wherein the subject carries a single copy of the APOE4 allele.

24. A kit according to claim 21 wherein the subject has been pre-determined to be APOE4−.

25. A kit according to claim 21 wherein the subject has been predetermined to carry a single copy of the APOE4 allele.

26. A method, PPAR-gamma agonist, use or kit according to claim 1 wherein the subject is suffering from MCI.

27. A method, PPAR-gamma agonist, use or kit according to claim 1, wherein the subject is suffering from Alzheimer's disease.

28. A method, PPAR-gamma agonist, use or kit according to claim 1, wherein the subject does not suffer from Type II diabetes.

29. A method, PPAR-gamma agonist, use or kit according to claim 1, wherein the subject does not suffer from Type II diabetes.

30. A method, PPAR-gamma agonist, use or kit according to claim 1, wherein the PPAR-gamma agonist is farglitazar.

31. A method, PPAR-gamma agonist, use or kit according to claim 1, wherein the PPAR-gamma agonist is a thiazolidinedione.

32. A method, PPAR-gamma agonist, use or kit according to claim 31, wherein the thiazolidinedione is pioglitazone.

33. A method, PPAR-gamma agonist, use or kit according to claim 31, wherein the thiazolidinedione is rosiglitazone.

34. A method, PPAR-gamma agonist, use or kit according to claim 33, wherein the rosiglitazone is in the form of rosiglitazone maleate.

35. A method, PPAR-gamma agonist, use or kit according to claim 33, wherein the rosiglitazone is provided at a dosage level of between 0.01 mg to 12 mg daily.

36. A method, PPAR-gamma agonist, use or kit according to claim 35, wherein the rosiglitazone is provided at a dosage level of 2 mg, 4 mg or 8 mg daily.

37. A method, PPAR-gamma agonist, use or kit according to claim 35, wherein the rosiglitazone is provided at a dosage level of 8 mg or more daily.

38. A method, PPAR-gamma agonist, use or kit according to claim 1, wherein the PPAR-gamma agonist is presented as an extended release formulation.

39. A method, PPAR-gamma agonist, use or kit according to claim 1, wherein the PPAR-gamma agonist is presented as an extended release tablet comprising a core which contains a depot of an immediate release formulation and a depot of a modified release formulation.

40. A method, PPAR-gamma agonist, use or kit according to claim 39 wherein the tablet is surrounded by a coating through which holes penetrate; at least one penetrating to the immediate release depot and at least one penetrating to the modified release depot.

41. A method, PPAR-gamma agonist, use or kit according to claim 1, wherein the PPAR-gamma agonist is presented in a form suitable for administration as a single daily dose.

42. A method, PPAR-gamma agonist, use or kit according to claim 1, wherein the PPAR-gamma agonist is administered in combination with a further medicament for the treatment or prevention of Alzheimer's disease or other dementias.

43. A method, PPAR-gamma agonist, use or kit according to claim 42, wherein the further medicament is a cholinesterase inhibitor.

44. A method, PPAR-gamma agonist, use or kit according to claim 43, wherein the cholinesterase inhibitor is tacrine, galantamine, rivastigamine or donepezil.

45. A method, PPAR-gamma agonist, use or kit according to claim 42, wherein the further medicament is an NMDA receptor antagonist.

46. A method, PPAR-gamma agonist, use or kit according to claim 45, wherein the NMDA receptor antagonist is memantine.

47. A method, PPAR-gamma agonist, use or kit according to claim 42, wherein the further medicament is a non-steroidal anti-inflammatory drug.

48. A method, PPAR-gamma agonist, use or kit according to claim 47, wherein the non-steroidal anti-inflammatory drug is naproxen, ibuprofen, diclofenac, indomethacin, nabumetone, piroxicam, celecoxib or asprin.

49. A method according to claim 1, wherein the screening to determine that the subject is not homozygous for the APOE4 allele comprises the use of a PCR-based method.

50. A method for improving cognitive function in a subject suffering from MCI or Alzheimer's disease, which subject is not homozygous for the APOE4 allele, comprising the steps of:
(i) screening the subject to determine that the subject is not homozygous for the APOE4 allele; and then
(ii) administering a safe and effective amount of a PPAR-gamma agonist to said subject.
